SEQUENCE LISTING (13 sequences)

SEQ ID NO: 1 - DNA, 9885 bp, Artificial Sequence
  vector
ccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacaaattacaacggtatatatcctgccagtactcggccgtcgacctgcaggaattctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctaactataacggtcctaaggtagcgaccgcgggacaacgggcccgtcgactgcagagggtagcgatcgccatggagccatttacaattgaatatatcctgccg

SEQ ID NO: 2 - DNA, 11344 bp, Artificial Sequence
  vector
cagtactcggccgtcgacctgcaggcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagaacgcgtgatctcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtcctctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttatagagagagagatagatttaattaccctgttattagagagagactggtgatttcagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactgcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgacaaattacaacggtatatatcctgc

SEQ ID NO: 3 - DNA, 11343 bp, Artificial Sequence
  vector
acgcgacaaattacaacggtatatatcctgccagtactcggccgtcgacctgcaggcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagaacgcgtgatctcagatctcggtgacgggcaggaccggacggggcggtaccggcaggctgaagtccagctgccagaaacccacgtcattctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttatagagagagagatagatttaattaccctgttattagagagagactggtgatttcagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactgcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcac

SEQ ID NO: 4 - DNA, 11340 bp, Artificial Sequence
  vector
aattacaacggtatatatcctgccagtactcggccgtcgacctgcaggcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagaacgcgtgatctcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttatagagagagagatagatttaattaccctgttattagagagagactggtgatttcagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactgcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgaca

SEQ ID NO: 5 - DNA, 11328 bp, Artificial Sequence
  vector
aattacaacggtatatatcctgccagtactcggccgtcgacctgcaggcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagaacgcgtgatctcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccatctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttatagagagagagatagatttaattaccctgttattagagagagactggtgatttcagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactgcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgaca

SEQ ID NO: 6 - DNA, 11290 bp, Artificial Sequence
  vector
aattacaacggtatatatcctgccagtactcggccgtcgacctgcaggcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagaacgcgtgatctcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctctctagatatcggatccccaagacgaattcgaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtctcgagatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgctatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggatatcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatctgcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggttatagagagagagatagatttaattaccctgttattagagagagactggtgatttcagcgtgtcctctccaaatgaaatgaacttccttatatagaggaagggtcttgcgaaggatagtgggattgtgcgtcatcccttacgtcagtggagatgtcacatcaatccacttgctttgaagacgtggttggaacgtcttctttttccacgatgctcctcgtgggtgggggtccatctttgggaccactgtcggcagaggcatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttggtcttctgagactgtatctttgacatttttggagtagaccagagtgtcgtgctccaccatgttgacgaagattttcttcttgtcattgagtcgtaaaagactctgtatgaactgttcgccagtcttcacggcgagttctgttagatcctcgatttgaatcttagactccatgcatggccttagattcagtaggaactacctttttagagactccaatctctattacttgccttggtttatgaagcaagccttgaatcgtccatactgcgatcgccatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaagtgctgtgcacggatctgccctggcttcaggagatcggaagacctcggccgtccgggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcagggatcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcggataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcggcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgaca

SEQ ID NO: 7 - DNA, 1671 bp, Artificial Sequence
  bar cassette
ccgcgttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataaccatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacgcgtctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 8 - DNA, 4618 bp, Artificial Sequence
  vector
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacggccgtcaaggccaagcttcccacttatctagaccgcgttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctaataacagggtaattaaatctatctctctctctataaccatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgcgatatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacgcgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcctgcaggtaagtgggatatcacgtgaagcttgcaagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - DNA, 22 bp, Artificial Sequence
  primer
ccaggagatcaaataccttccc

SEQ ID NO: 10 - DNA, 23 bp, Artificial Sequence
  primer
atcatcgcaagaccggcaacagg

SEQ ID NO: 11 - DNA, 21 bp, Artificial Sequence
  primer
aacagcggtcattgactggag

SEQ ID NO: 12 - DNA, 24 bp, Artificial Sequence
  primer
gagtgagaattgacgggatctatg

SEQ ID NO: 13 - DNA, 24 bp, Artificial Sequence
  primer
attgccaaatgtttgaacgatctg

(19) United States
(12) Patent Application Publication
(10) Pub. No.: US 2016/0053274 A1
(43) Pub. Date: Feb. 25, 2016

(54) TARGETED GENOME ENGINEERING IN EUKARYOTES

(71) Applicant: BAYER CROPSCIENCE NV, B-Diegem (BE)

(72) Inventor: Katelijn D'HALLUIN, Mariakerke (BE)

(21) Appl. No.: 14/782,238

(22) PCT Filed: Mar. 31, 2014
(86) PCT No.: PCT/EP14/56467

§ 371 Date: Oct. 2, 2015

(30) Foreign Application Priority Data
Apr. 2, 2013 (EP) 13161963.7

(51) Int. Cl. C12N 15/82

(52) U.S. Cl. CPC C12N 15/8213

(57) ABSTRACT

Improved methods and means are provided to modify in a targeted manner the genome of a eukaryotic cell at a predefined site using a double stranded break inducing enzyme such as a TALEN and a donor molecule for repair of the double stranded break.

### FIELD OF THE INVENTION

The invention relates to the field of agronomy. More particularly, the invention provides methods and means to introduce a targeted modification, including insertion, deletion or substitution, at a precisely localized nucleotide sequence in the genome of a eukaryotic cell, e.g. a plant cell. The modifications are triggered in a first step by induction of a double stranded break at a recognition nucleotide sequence using a double stranded DNA break inducing enzyme, e.g. a TALEN, while a repair nucleic acid molecule is subsequently used as a template for introducing a genomic modification at or near the cleavage site by homologous recombination. The frequency of targeted insertion events is increased when designing the sequences of the repair DNA that mediated the homologous recombination to target insertion outside the cleavage and recognition site as compared to precisely at the cleavage site.

### BACKGROUND

The need to introduce targeted modifications in genomes, such a plant genomes, including the control over the location of integration of foreign DNA has become increasingly important, and several methods have been developed in an effort to meet this need (for a review see Kumar and Fladung, 2001, Trends in Plant Science, 6, pp 155-159). These methods mostly rely on the initial introduction of a double stranded DNA break at the targeted location via expression of a double strand break inducing (DSBI) enzyme.

Activation of the target locus and/or repair or donor DNA through the induction of double stranded DNA breaks (DSB) via rare-cutting endonucleases, such as I-Scel has been shown to increase the frequency of homologous recombination by several orders of magnitude. (Puchta et al., 1996, Proc. Natl. Acad. Sci. U.S.A., 93, pp 5055-5060; Chilton and Que, Plant Physiol., 2003; D'Halluin et al. 2008 Plant Biotechnol. J. 6, 93-102).

WO 2005/049842 describes methods and means to improve targeted DNA insertion in plants using rare-cleaving “double stranded break” inducing (DSBI) enzymes, as well as improved I-Scel encoding nucleotide sequences.

WO2006/105946 describes a method for the exact exchange in plant cells and plants of a target DNA sequence for a DNA sequence of interest through homologous recombination, whereby the selectable or screenable marker used during the homologous recombination phase for temporal selection of the gene replacement events can subsequently be removed without leaving a foot-print and without resorting to in vitro culture during the removal step, employing the therein described method for the removal of a selected DNA by microspore specific expression of a DSBI rare-cleaving endonuclease.

WO2008/037436 describe variants of the methods and means of WO2006/105946 wherein the removal step of a selected DNA fragment induced by a double stranded break inducing rare cleaving endonuclease is under control of a germline-specific promoter. Other embodiments of the method relied on non-homologous end-joining at one end of the repair DNA and homologous recombination at the other end. WO08/148559 describes variants of the methods of WO2008/037436, i.e. methods for the exact exchange in eukaryotic cells, such as plant cells, of a target DNA sequence for a DNA sequence of interest through homologous recombination, whereby the selectable or screenable marker used during the homologous recombination phase for temporal selection of the gene replacement events can subsequently be removed without leaving a foot-print employing a method for the removal of a selected DNA flanked by two nucleotide sequences in direct repeats.

In addition, methods have been described which allow the design of rare cleaving endonucleases to alter substrate or sequence-specificity of the enzymes, thus allowing to induce a double stranded break at a locus of interest without being dependent on the presence of a recognition site for any of the natural rare-cleaving endonucleases. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA 94, 5525-5530). Another way of producing custom-made meganucleases, by selection from a library of variants, is described in WO2004/067736. Custom made meganucleases or redesigned meganucleases with altered sequence specificity and DNA-binding affinity may also be obtained through rational design as described in WO2007/047859. Further, WO10/079430, and WO11/072246 describe the design of transcription activator-like effectors (TALEs) proteins with customizable DNA binding specificity and how these can be fused to nuclease domains (e.g. FOKI) to create chimeric restriction enzymes with sequence specificity for basically any DNA sequence, i.e. TALE nucleases (TALENs).

Bedell et al., 2012 (Nature 491:p 114-118) and Chen et al., 2011 (Nature Methods 8:p 753-755) describe oligo-mediated genome editing in mammalian cells using TALENs and ZFNs respectively.

Elliot et al (1998, Mol Cel Biol 18:p 93-101) describes a homology-mediated DSB repair assay wherein the frequency of incorporation of mutations was found to inversely correlate with the distance from the cleavage site.

WO11/154158 and WO11/154159 describe methods and means to modify in a targeted manner the plant genome of transgenic plants comprising chimeric genes wherein the chimeric genes have a DNA element commonly used in plant molecular biology, as well as re-designed meganucleases to cleave such an element commonly used in plant molecular biology.

PCT/EP12/065867 describes methods and means are to modify in a targeted manner the genome of a plant in close proximity to an existing elite event using a double stranded DNA break inducing enzyme.

However, there still remains a need for optimizing the enzymes and repair molecules and their use to enhance the efficiency, accuracy and specificity of targeted genome engineering. The present invention provides an improved method for making targeted sequence modifications, such as insertions, deletions and replacements, as will be described hereinafter, in the detailed description, examples and claims.

### SUMMARY

In a first embodiment, the invention provides a method for modifying the genome of a eukaryotic cell at a preselected site comprising the steps of:
a. Inducing a double stranded DNA break (DSB) in the genome of said cell at a cleavage site at or near a recognition site for a double stranded DNA beak inducing (DSBI) enzyme by expressing in said cell a DSBI enzyme recognizing said recognition site and inducing a DSB at said cleavage site;
b. Introducing into said cell a repair nucleic acid molecule comprising an upstream flanking region having homology to the region upstream of said preselected site and/or a downstream flanking region having homology to the DNA region downstream of said preselected site for allowing homologous recombination between said flanking region or regions and said DNA region or regions flanking said preselected site;
c. Selecting a cell having a modification of said genome at said preselected site selected from
   i. a replacement of at least one nucleotide;
   ii. a deletion of at least one nucleotide;
   iii. an insertion of at least one nucleotide; or
   iv. any combination of i.-iii.
characterised in that said preselected is located outside said cleavage and/or recognition site.

The preselected site should not overlap with the cleavage and/or recognition site. Accordingly, the preselected site, or the most proximal nucleotide thereof, may be located at least 25 bp from the cleavage site, such as at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site. On other words, 3′ end of the upstream flanking region should align at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp or at least 500 bp away from the cleavage site, and/or the 5′-end of the downstream flanking region should align at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site.

In an even further embodiment, the DSBI enzyme creates a 5′ overhang upon inducing said DSB, such as a DSBI enzyme with a FOKI catalytic domain (e.g. a TALEN or ZFN). In another embodiment, the DSBI enzyme functions as a dimer, wherein the two monomers bind to distinct domains within the total recognition sequence, such as a TALEN or a ZFN. In another embodiment, the DSBI enzyme can be a TALEN, for example a TALEN with a FOKI catalytic domain.

In a further embodiment, the repair molecule also comprises a recognition and cleavage site for the DSBI enzyme, preferably in one of the flanking regions. The repair molecule may be a double stranded DNA molecule. The repair molecule may also comprises a nucleic acid molecule of interest, which is being inserted at the preselected through homologous recombination between the flanking DNA region or regions and said DNA region or regions flanking the preselected site, optionally in combination with non-homologous end-joining. The nucleic acid molecule of interest may comprise one or more expressible gene(s) of interest, such as herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites. The nucleic acid molecule of interest may also comprise a selectable or screenable marker gene.

The modification of the genome at the preselected site may be a replacement or insertion, such as a replacement or insertion of at least 43 nucleotides.

The DSBI enzyme can be expressed in said cell by introducing into the cell a nucleic acid molecule encoding that DSBI enzyme.

In a further embodiment, the eukaryotic cell is a plant cell.

The preselected site can be located in the flanking region of an elite event.

The eukaryotic cell, such as a plant cell, can further be grown into a eukaryotic organism, such as a plant.

Also provide is the use of a DSBI enzyme (in combination with a repair nucleic acid molecule comprising at least one flanking region), such as a DSBI enzyme creating a 5′ overhang upon cleavage, or a TALEN, or a ZFN, to modify the genome at a preselected site located outside the cleavage and/or recognition site of said DSBI enzyme.

In another aspect, the invention provides a method for increasing the mutation frequency at a preselected site of the genome of a eukaryotic cell comprising the steps of:
a. Inducing a double stranded DNA break (DSB) in the genome of said cell at a cleavage site at or near a recognition site for a double stranded DNA beak inducing (DSBI) enzyme by expressing in the cell a DSBI enzyme recognizing the recognition site and inducing a DSB at the cleavage site;
b. Introducing into the cell a foreign nucleic acid molecule;
c. Selecting a cell wherein the DSB has been repaired, the repair of the DSB resulting in a modification of said genome at said preselected site, wherein the modification is selected from;
   i. a replacement of at least one nucleotide;
   ii. a deletion of at least one nucleotide;
   iii. an insertion of at least one nucleotide; or
   iv. any combination of i.-iii.
characterised in that the foreign nucleic acid molecule also comprises a recognition site and cleavage site for the DSBI enzyme.

In this aspect, the foreign nucleic acid molecule may comprise a nucleotide sequence of at least 20 nt in length having at least 80% sequence identity to a genomic DNA region within 5000 bp of said recognition and cleavage site.

Further provided is a eukaryotic cell or eukaryotic organism, such as a plant cell or plant, comprising a modification at a predefined site of the genome, obtainable by any of the preceding methods.

The invention also provides a method for producing a plant comprising a modification at a predefined site of the genome, comprising the step of crossing a plant obtainable by any of the preceding methods with another plant or with itself and optionally harvesting seeds.

Also provided is a method of growing a plant obtainable by any of the preceding methods, comprising the step of applying a chemical to said plant or substrate wherein said plant is grown, a process of growing a plant in the field comprising the step of applying a chemical compound on a plant obtainable by any of the preceding methods, a process of producing treated seed comprising the step applying a chemical compound on a seed of plant obtainable by any of the preceding methods, and a method for producing feed, food or fiber comprising the steps of providing a population of plants obtainable by any of the preceding methods and harvesting seeds.

### FIGURE LEGENDS

FIG. 1: Schematic representation of mutation induction at a TALEN cleavage site in the presence of a foreign DNA molecule with or without flanking regions comprising the TALEN recognition and cleavage site as described in Example 3. Scissors indicate TALEN cleavage at nucleotide position 86 and 334 of the bar coding region (horizontally striped box) respectively. Foreign DNA molecules (in this cases used for selection of transformed events) comprise a hygromycin-expression cassette either flanked by sequences homologous to the bar gene flanking position 140 (pTCV224) or 479 (PTCV225) or not flanked by homologous sequences (pTIB235). Transformants are selected for hyg-resistance and subsequently screened for PPT-sensitivity, indicative for an inactivating mutation in the bar gene.

FIG. 2: Schematic representation of targeted sequence insertion (TSI) at a TALEN cleavage site or within the TALEN recognition site of repair DNA molecules wherein the flanking regions do or do not comprise (parts of) the half part TALEN recognition sites, as described in Example 4 (first part). Scissors indicate TALEN cleavage at nucleotide position 334 of the bar coding region (horizontally striped box), with a magnification of the TALEN recognition site, comprised of two half part binding sites (white boxes) and a spacer region (checkered box). All three repair DNA vectors comprise flanking regions corresponding to the regions flanking the bar gene at position 334 (horizontally striped boxes) as indicated, pJR21 exactly flanking position 334 and thus containing sequences corresponding to both the half-part binding sites (white boxes) and spacer region (checkered boxes), pJR23 lacking the sequences corresponding to spacer region but containing sequences corresponding the binding sites region (white boxes), and pJR25 lacking the entire TALEN recognition site. The location of the primers used for identification of TSI events is indicated by the thick black arrows, the length of the corresponding PCR fragments by the two-sided arrows below. The asterisks at the repair DNA vectors indicate a truncation of the 35S promoter by which it can no longer be recognized by primer IB448, thereby allowing the unequivocal identification of the insertion of the hyg cassette at the target locus.

FIG. 3: Schematic representation of targeted sequence insertion (TSI) away from the TALEN cleavage site of a repair DNA molecules wherein the flanking regions of the repair DNA target insertion of the hyg-cassette either upstream or downstream of the cleavage site, as described in Example 4 (second part). Scissors indicate TALEN cleavage at nucleotide position 86 and 334 of the bar coding region (horizontally striped box) respectively. Repair DNA pTCV224 comprises flanking region corresponding to nt 1-144 and 141-552 of the bar gene respectively, resulting in an insertion of the hyg-cassete at position 144 while repair DNA pTCV225 comprises flanking regions corresponding to nt 1-479 and 476-552 of the bar gene respectively, resulting in an insertion of the hyg-cassete at position 479. The location of the primers used for identification of TSI events is indicated by the thick black arrows, the length of the corresponding PCR fragments by the two-sided arrows below. The asterisks at the repair DNA vectors indicate a truncation of the 35S promoter such that it can no longer be recognized by primer IB448, thereby allowing the unequivocal identification of the insertion of the hyg cassette at the target locus.

FIG. 4: Footprint over the TALEN cleavage site: Alignment of TALENbar334-pTCV225 TSI events at the cleavage site. The upper sequence is the unmodified pTCV225 sequence and below the various identified TSI events (see also table 5). The spacer region is boxed and the two half-part binding sites (BS1 and BS2) of the TALENbar334 are underlined.

FIG. 5: Schematic representation of allele surgery away from the TALEN cleavage site using a repair DNA wherein the flanking regions target insertion of a GA dinucleotide at position 169 of the bar gene, as described in Example 5. Scissors indicate TALEN cleavage at nucleotide position 86 and 334 of the bar coding region (horizontally striped box) respectively. Repair DNA pJR19 comprises flanking region corresponding to nt 1-169 and 170-552 of the bar gene respectively, resulting in an insertion of a GA at position 169. This insertion creates a premature stop codon as well as an EcoRV site. The location of the primers used for identification of recombination events is indicated by the thick black arrows, the length of the corresponding PCR fragments by the two-sided arrows below. Primer AR35 is specific for the nos termination, present in both the genome of the target line as well as the repair DNA. As the pJR19 plasmid contained the entire 35S promoter, a primer specific for the genomic target (AR32) was used to identify targeted insertion events from non-targeted ones. The obtained PCR product is subsequently cleaved with EcoRV to determine correct insertion of the GA.

### DETAILED DESCRIPTION

The inventors have found that when designing the repair DNA molecule for homology-mediated repair of a TALEN-induced genomic double stranded DNA break (DSB) in such a way that the flanking regions do not correspond to the DNA regions immediately flanking the genomic cleavage site, targeted sequence insertion (TSI) is enhanced, for example when no sequences corresponding to the cleavage site and recognition site were included in the flanking regions. Secondly, it was found that when designing the flanking regions of the repair DNA molecule so as to target insertion further away from the cleavage site instead of at or surrounding the cleavage site, homology-mediated targeted sequence insertion (TSI) is unexpectedly further increased by 2-4-fold. This reduces the need to specifically design repair molecules for each DSBI enzyme that is evaluated for cleavage at a particular locus, while on the other hand allowing multiple modifications to be made at a certain locus using only one enzyme in combination with various repair molecules. In addition, the genomic DSB which is often repaired by NHEJ, results in basically a unique fingerprint allowing discrimination and tracing of each generated event. Finally, the inventors have demonstrated that DSBI-enzyme mediated mutation induction at a preselected site of the genome was remarkably enhanced in the presence of a foreign DNA molecule that also contained a recognition site for the DSBI enzyme (and hence could also be cleaved by the DSBI enzyme).

Thus, in a first aspect, the invention relates to a method for modifying the genome, preferably the nuclear genome, of a eukaryotic cell at a preselected site comprising the steps of:
a. Inducing a double stranded DNA break (DSB) in the genome of said cell at a cleavage site at or near a recognition site for a double stranded DNA break inducing (DSBI) enzyme by expressing in said cell a DSBI enzyme recognizing said recognition site and inducing said DSB at said cleavage site;
b. Introducing into said cell a repair nucleic acid molecule comprising an upstream flanking region having homology to the DNA region upstream of said preselected site and/or a downstream flanking DNA region having homology to the DNA region downstream of said preselected site for allowing homologous recombination between said flanking region or regions and said DNA region or regions flanking said preselected site;
c. Selecting a cell wherein said repair nucleic acid molecule has been used as a template for making a modification of said genome at said preselected site, wherein said modification is selected from
   i. a replacement of at least one nucleotide;
   ii. a deletion of at least one nucleotide;
   iii. an insertion of at least one nucleotide; or
   iv. any combination of i.-iii.
characterised in that said preselected site is located outside or away from said cleavage (and/or recognition) site or wherein said preselected site does not comprise said cleavage site and/or recognition site.

As used herein, a “double stranded DNA break inducing enzyme” is an enzyme capable of inducing a double stranded DNA break at a particular nucleotide sequence, called the “recognition site”. Rare-cleaving endonucleases are DSBI enzymes that have a recognition site of about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level.

A list of other rare cleaving meganucleases and their respective recognition sites is provided in Table I of WO 03/004659 (pages 17 to 20) (incorporated herein by reference). These include I-Sce I, I-Chu I, I-Dmo I, I-Cre I, I-Csm I, PI-Fli I, Pt-Mtu I, I-Ceu I, I-Sce II, I-Sce III, HO, PI-Civ I, PI-Ctr I, PI-Aae I, PI-BSU I, PI-Dhal, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I or PI-Tsp I.

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA 94, 5525-5530). Custom-made meganucleases can be produced by selection from a library of variants, is described in WO2004/067736. Custom made meganucleases with altered sequence specificity and DNA-binding affinity may also be obtained through rational design as described in WO2007/047859. Another example of custom-designed endonucleases include the so-called TALE nucleases (TALENs), which are based on transcription activator-like effectors (TALEs) from the bacterial genus Xanthomonas fused to the catalytic domain of a nuclease (e.g. FOKI). The DNA binding specificity of these TALEs is defined by repeat-variable diresidues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e.g. Boch et al., 2009, Science 326:p 1509-1512; Moscou and Bogdanove, 2009, Science 326:p 1501; Christian et al., 2010, Genetics 186:p 757-761; and WO10/079430, WO11/072246, WO2011/154393, WO11/146121, WO2012/001527, WO2012/093833, WO2012/104729, WO2012/138927, WO2012/138939). WO2012/138927 further describes monomeric (compact) TALENs and TALENs with various catalytic domains and combinations thereof. Recently, a new type of customizable endonuclease system has been described; the so-called CRISPR/Cas system, which employs a special RNA molecule (crRNA) conferring sequence specificity to guide the cleavage of an associated nuclease Cas9 (Jinek et al, 2012, Science 337:p 816-821). Such custom designed rare-cleaving endonucleases are also referred to as a non-naturally occurring rare-cleaving endonucleases.

The cleavage site of a DSBI enzyme relates to the exact location on the DNA where the double-stranded DNA break is induced. The cleavage site may or may not be comprised in (overlap with) the recognition site of the DSBI enzyme and hence it is said that the cleavage site of a DSBI enzyme is located at or near its recognition site. The recognition site of a DSBI enzyme, also sometimes referred to as binding site, is the nucleotide sequence that is (specifically) recognized by the DSBI enzyme and determines its binding specificity. For example, a TALEN or ZNF monomer has a recognition site that is determined by their RVD repeats or ZF repeats respectively, whereas its cleavage site is determined by its nuclease domain (e.g. FOKI) and is usually located outside the recognition site. In case of dimeric TALENs or ZFNs, the cleavage site is located between the two recognition/binding sites of the respective monomers, this intervening DNA region where cleavage occurs being referred to as the spacer region. For meganucleases on the other hand, DNA cleavage is effected within its specific binding region and hence the binding site and cleavage site overlap.

A person skilled in the art would be able to either choose a DSBI enzyme recognizing a certain recognition site and inducing a DSB at a cleavage site at or in the vicinity of the preselected site or engineer such a DSBI enzyme. Alternatively, a DSBI enzyme recognition site may be introduced into the target genome using any conventional transformation method or by crossing with an organism having a DSBI enzyme recognition site in its genome, and any desired DNA may afterwards be introduced at or in the vicinity of the cleavage site of that DSBI enzyme.

As used herein, a repair nucleic acid molecule, is a single-stranded or double-stranded DNA molecule or RNA molecule that is used as a template for modification of the genomic DNA at the preselected site in the vicinity of or at the cleavage site. As used herein, “use as a template for modification of the genomic DNA”, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

As used herein, “a modification of the genome”, means that the genome has changed by at least one nucleotide. This can occur by replacement of at least one nucleotide and/or a deletion of at least one nucleotide and/or an insertion of at least one nucleotide, as long as it results in a total change of at least one nucleotide compared to the nucleotide sequence of the preselected genomic target site before modification, thereby allowing the identification of the modification, e.g. by techniques such as sequencing or PCR analysis and the like, of which the skilled person will be well aware.

As used herein “a preselected site” or “predefined site” indicates a particular nucleotide sequence in the genome (e.g. the nuclear genome) at which location it is desired to insert, replace and/or delete one or more nucleotides. This can e.g. be an endogenous locus or a particular nucleotide sequence in or linked to a previously introduced foreign DNA or transgene. The preselected site can be a particular nucleotide position at(after) which it is intended to make an insertion of one or more nucleotides. The preselected site can also comprise a sequence of one or more nucleotides which are to be exchanged (replaced) or deleted.

As used herein, a flanking region, is a region of the repair nucleic acid molecule having a nucleotide sequence which is homologous to the nucleotide sequence of the DNA region flanking (i.e. upstream or downstream) of the preselected site. It will be clear that the length and percentage sequence identity of the flanking regions should be chosen such as to enable homologous recombination between said flanking regions and their corresponding DNA region upstream or downstream of the preselected site. The DNA region or regions flanking the preselected site having homology to the flanking DNA region or regions of the repair molecule are also referred to as the homology region or regions in the genomic DNA.

To have sufficient homology for recombination, the flanking DNA regions of the repair nucleic acid molecule may vary in length, and should be at least about 10, about 15 or about 20 nt in length. However, the flanking region may be as long as is practically possible (e.g. up to about 100-150 kb such as complete bacterial artificial chromosomes (BACs). Preferably, the flanking region will be about 50 nt to about 2000 nt, e.g. about 100 nt, 200 nt, 500 nt or 1000 nt. Moreover, the regions flanking the DNA of interest need not be identical to the homology regions (the DNA regions flanking the preselected site) and may have between about 80% to about 100% sequence identity, preferably about 95% to about 100% sequence identity with the DNA regions flanking the preselected site. The longer the flanking region, the less stringent the requirement for homology. Furthermore, to achieve exchange of the target DNA sequence at the preselected site without changing the DNA sequence of the adjacent DNA sequences, the flanking DNA sequences should preferably be identical to the upstream and downstream DNA regions flanking the preselected site.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

In order to target sequence modification at the preselected site, the flanking regions must be chosen so that 3′ end of the upstream flanking region and/or the 5′ end of the downstream flanking region align(s) with the ends of the predefined site. As such, the 3′ end of the upstream flanking region determines the 5′ end of the predefined site, while the 5′ end of the downstream flanking region determines the 3′ end of the predefined site.

As used herein, said preselected site being located outside or away from said cleavage (and/or recognition) site, means that the site at which it is intended to make the genomic modification (the preselected site) does not comprise the cleavage site and/or recognition site of the DSBI enzyme, i.e. the preselected site does not overlap with the cleavage (and/or recognition) site. Outside/away from in this respect thus means upstream or downstream of the cleavage (and/or recognition) site. This can be e.g. at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site. When the preselected site comprises one or more nucleotides that are to be exchanged or deleted, the distance from the cleavage site is relative to the most proximal nucleotide of the preselected site, i.e. the 5′ or 3′ end of the preselected site, depending on the relative orientation of the preselected site with respect to the cleavage site. Thus the most proximal nucleotide of the preselected site should be located at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site.

In terms of the flanking regions, the preselected site being located outside or away from the cleavage site thus means that the 3′ end of the upstream flanking region aligns at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp or at least 500 bp away from the cleavage site, and/or that the 5′-end of the downstream flanking region aligns at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site.

In terms of the homology regions in the genomic DNA, the preselected site being located outside or away from the cleavage site thus means that the cleavage site (and recognition site) is not located between the upstream and downstream homology regions. The cleavage site (and recognition site) should be located within one of the homology regions or even outside of the homology regions.

For example, the 3′ end of the upstream flanking region of repair DNA vector pTCV224 aligns 58 bp downstream from the TALENbar86 cleavage site and 190 bp upstream from the TALENbar334 cleavage site, while the 5′ end of the downstream flanking region of pTCV224 aligns 55 bp downstream from the TALENbar86 cleavage site and 193 bp upstream from the TALENbar334 cleavage site leading to an insertion of the DNA region between the flanking regions (the nucleic acid molecule of interest) at a position 55-58 bp downstream of or 190-193 bp upstream of the respective cleavage sites. Likewise, the 3′ end of the upstream flanking region of repair DNA vector pTCV225 aligns 393 bp downstream from the TALENbar86 and 145 bp downstream from the TALENbar334 cleavage site, while the 5′ end of the downstream flanking region of pTCV225 aligns 390 bp downstream from the TALENbar86 cleavage site and 142 bp downstream from the TALENbar334 cleavage site, leading to an insertion of the DNA region between the flanking regions (the nucleic acid molecule of interest) at a position 390-393 bp or 142-145 bp downstream of the respective cleavage sites.

It will be understood that in order to induce modification of the genome at the preselected site by the repair nucleic acid molecule, preselected site or at least the most proximal nucleotide thereof should also not be located too far away from the cleavage site but they must be located in the vicinity of each other. The most proximal nucleotide of the preselected site should be located between about 25-5000 bp from the cleavage site, such as between about 30-2500 bp, between about 50-1000 bp, between about 50-500 bp or between about 100-500 bp from the cleavage site (either upstream or downstream). Relating to the flanking regions, the 3′ end of the upstream flanking region and/or the 5′ end of the downstream flanking region must align between about 25-5000 bp from the cleavage site, such as between about 30-2500 bp, between about 50-1000 bp, between about 50-500 bp or between about 100-500 bp from the cleavage site (upstream or downstream).

Eukaryotic cells make use of various mechanisms to repair double stranded DNA break, as reviewed in e.g. Mimitou et al., (2009, Trends Biol Sci 34: p 264-272) and Blackwood et al. (2013, Biochem. Soc Transactions, 41:314-320), the main ones being none-homologous end-joining (NHEJ) and homologous recombination. NHEJ is fast and efficient, but highly error prone and hence often leads to small mutations. Homologous recombination starts by so-called-end resection, which involves the 5′-3′ degradation of the generated DNA ends to create a 3′ single-stranded overhang by various 5′-3′ exonucleases, ssDNA endonucleases and helicases. These 3′ single stranded ends are subsequently bound by ss-DNA binding proteins (e.g. Rad51), after which the thus generated nucleoprotein complex searches a second DNA molecule for homology, resulting in a pairing to the complementary strand in the homologous molecule. This process is referred to as strand invasion. The invading strand is then extended by DNA polymerisation using the donor molecule as a template. For the subsequent steps two models have been proposed. Following the synthesis-dependent strand annealing (SDSA) model, the invading strand is displaced and pairs with the other single stranded tail, allowing DNA synthesis to complete repair. Following the DSB repair (DSBR) model, the other end of the break is captured by the displaced strand from the donor duplex (D-loop) and is used to prime a second round of leading strand DNA synthesis. A double Holliday junction (dHJ) intermediate is then formed which can be resolved to form either a crossover or a non-crossover products (Mimitou et al., supra). It has been suggested that in Drosophila homologous replacement occurs via both models (Carol) et al, 2012, Genetics 118:p 773-782).

Meganucleases, in particular LAGLIDADG meganucleases, mostly generate 3′ overhangs (Chevalier and Stoddar, 2001, Nucleic Acids Res 29(18): 3757-74), for an overview see Hafez and Hausner, 2002, Genome 55: p 553-569), and scarless relegation via NHEJ of meganuclease-induced DSB has been reported frequently (for an overview, see WO12/138927, p 36). Cas9 induces blunt ended DNA breaks (Choo et al., 2013, Nature Biotechn, ePub 29 January). Conventional ZFNs and TALENs, at least in as far as containing a FOKI catalytic domain, generate 5′ overhangs. This may influence the break repair process, which involves the generation of 3′ overhangs. In this way, 5′ overhang creating enzymes such as most TALENs may be more favourable for certain applications like sequence replacements, whereas for other applications like precise insertion meganucleases may be the DSBI enzyme of choice.

Accordingly, in one embodiment, the DSBI enzyme upon cleavage creates a 5′ overhang at its cleavage site. For avoidance of doubt, a 5′ overhang means that the 5′ end of the DNA strands making up a double stranded DNA at the cleavage site are at least one nucleotide longer than the 3′ end of the two strands. A 3′ overhang on the other hand means that the 3′ end of the DNA strands making up a double stranded DNA at the cleavage site are at least one nucleotide longer than the 5′ ends of the two strands. Both 3′ and 5′ overhangs are referred to as sticky ends, as opposed to blunt ends, where both strands are of the same length. The skilled person would be able to choose restriction enzymes creating 5′ overhangs. Information on commonly used restriction enzymes and their types of overhang can for example be found in (Brown. T. A. Molecular Biology LabFax: Recombinant DNA) and via http://rebase.neb.com/rebase/rebase.html. Catalytic domains of any such enzymes could be fused to any DNA binding moiety such as ZFs or TALEs to generated custom-designed rare-cleaving DSBI enzymes generating 5′ overhangs.

Using the present TALENs, it was observed that insertion at one side (in this case downstream with respect to the transcriptional direction of the bar coding region) of the break resulted in an increased frequency of TSI events, whereas insertion at the other side (in this case upstream with respect to the transcriptional direction of the bar coding region) of the break resulted in a decrease of TSI events. Without intending to limit the invention, it is believed this may be attributed to the properties of the two TALEN monomers constituting the functional dimeric enzyme. For example, the binding properties of the two monomers may differ such that one of the two molecules is more likely to remain bound to the genomic DNA and/or repair molecule at the time of recombination, thereby potentially posing sterical hindrance for the recombination process at one side of the break but not the other. As a result, non-homologous end-joining rather than homologous recombination may take place, leading to small mutations at the junction between the genomic DNA and the repair molecule. Whether insertion at either one or the other side of the break provides the best recombination frequency for a given DSBI enzyme can easily be experimentally determined.

Thus, in another embodiment, the DSBI enzyme functions as a dimer, whereby the two monomers constituting the dimer bind to distinct parts of the total recognition site of the dimeric enzyme. This is the case for e.g. TALENs and ZFNs, where each monomer binds one half-part recognition site.

In a further embodiment, the repair nucleic acid molecule also comprises a recognition and cleavage site for the DSBI enzyme, for example in one of the flanking regions, by designing the flanking region to overlap with the genomic DNA region containing the recognition site, such that the repair nucleic acid molecule can also be cleaved by the DSBI enzyme inducing the genomic break. It is believed that due to the presence of such a site in the repair nucleic acid molecule, the repair nucleic acid molecule is also cleaved by the DSBI enzyme, resulting in an increased in recruitment of cellular proteins involved in DNA repair. As a consequence of this recruitment, there is a more efficient repair of the genomic break and hence also a higher chance of incorporation of the repair nucleic acid molecule at the preselected site in the vicinity of the cleavage site.

In a specific embodiment, the repair nucleic acid molecule is a double stranded molecule, such as a double stranded DNA molecule.

In one embodiment, the repair nucleic acid molecule may consist of two flanking regions, i.e. both an upstream and a downstream flanking region but without any intervening sequences (without a nucleic acid molecule of interest), thereby allowing the deletion of DNA sequences at the preselected site that are located between the genomic homology regions.

In another embodiment, the repair nucleic acid molecule may further comprise a nucleic acid molecule of interest, which is inserted at the preselected site via homologous recombination between the upstream and/or downstream flanking region and the corresponding genomic DNA region(s) flanking the preselected site. In case of one flanking region, the nucleic acid molecule of interest may be inserted at the preselected site through a combination of homologous recombination at the side of the flanking region and non-homologous end-joining at the other end, and hence can be used for targeted sequence insertions. In case of two flanking regions the nucleic acid molecule of interest is located between the two flanking regions and depending on the design of the flanking regions is either inserted at the preselected site to result in an additional sequence being present or can be inserted such as to replace a genomic DNA sequence at the preselected site.

It will be clear that the methods according to the invention allow insertion of any nucleic acid molecule of interest including nucleic acid molecule comprising genes encoding an expression product (genes of interest), nucleic acid molecules comprising a nucleotide sequence with a particular nucleotide sequence signature e.g. for subsequent identification, or nucleic acid molecules comprising (inducible) enhancers or silencers, e.g. to modulate the expression of genes located near the preselected site.

In a particular embodiment, the nucleic acid molecule of interest is at least 25 nt in length, such as at least 43 nt, at least 50 nt, at least 75 nt, at least 100 nt, at least 150 nt, at least 200 nt, at least 250 nt at least 300 nt, at least 400 nt, at least 500 nt, at least 750 nt, at least 1 kb, at least 1.5 kb, at least 2 kb, at least 3 kb, at least 4 kb, at least 5 kb, at least 10 kb, at least 15 kb, at least 20 kb or even more. In this way, the introduced modification is a replacement or insertion of at least 25 nt, at least 43 nt, at least 50 nt, at least 75 nt, at least 100 nt, at least 150 nt, at least 200 nt, at least 250 nt at least 300 nt, at least 400 nt, at least 500 nt, at least 750 nt, at least 1 kb, at least 1.5 kb, at least 2 kb, at least 3 kb, at least 4 kb, at least 5 kb, or at least 10 kb, at least 15 kb, at least 20 kb or even more.

When the cell is a plant cell, the nucleic acid molecule of interest may also comprise one or more plant expressible gene(s) of interest, including but not limited to a herbicide tolerance gene, an insect resistance gene, a disease resistance gene, an abiotic stress resistance gene, an enzyme involved in oil biosynthesis or carbohydrate biosynthesis, an enzyme involved in fiber strength and/or length, an enzyme involved in the biosynthesis of secondary metabolites.

Herbicide-tolerance genes include a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024782. Glyphosate-tolerant plants can also be obtained by selecting plants containing naturally-occurring mutations of the above-mentioned genes, as described in for example WO 01/024615 or WO 03/013226. EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide tolerance genes may encode an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Phosphinothricin acetyltransferases are for example described in U.S. Pat. Nos. 5,561,236; 5,648,477; 5,646,024; 5,273,894; 5,637,489; 5,276,268; 5,739,082; 5,908,810 and 7,112,665.

Herbicide-tolerance genes may also confer tolerance to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, and WO 99/24586, WO 2009/144079, WO 2002/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 2004/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide tolerance genes encode variant ALS enzymes (also known as acetohydroxyacid synthase, AHAS) as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerance genes are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerance genes are described in for example WO 07/024782 and U.S. Patent Application No. 61/288,958.

Insect resistance gene may comprise a coding sequence encoding:

1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at:

http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302), or such proteins encoded by synthetic genes as e.g. described in and U.S. patent application Ser. No. 12/249,016; or

2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022 and EP 08010791.5); or

3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or

4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or

5) an insecticidal secreted protein from Bacillus thuringiensis or Bacillus cereus, or an insecticidal portion thereof, such as the vegetative insecticidal (VIP) proteins listed at:

http://www.lifesci.sussex.ac.uk/home/Neil_Crickmore/Bt/vip.html, e.g., proteins from the VIP3Aa protein class; or

6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or

7) a hybrid insecticidal protein comprising parts from different secreted proteins from Bacillus thuringiensis or Bacillus cereus, such as a hybrid of the proteins in 1) above or a hybrid of the proteins in 2) above; or

8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or

9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Appl. Nos. 61/126,083 and 61/195,019), or the binary toxin made up of the VIP3 protein and the Cry2Aa or Cry2Ab or Cry2Ae proteins (U.S. patent application Ser. No. 12/214,022 and EP 08010791.5);

10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein).

An “insect-resistant gene as used herein, further includes transgenes comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650.

Abiotic Stress Tolerance Genes Include

1) a transgene capable of reducing the expression and/or the activity of poly(ADP-ribose) polymerase (PARP) gene in the plant cells or plants as described in WO 00/04173, WO/2006/045633, EP 04077984.5, or EP 06009836.5.

2) a transgene capable of reducing the expression and/or the activity of the PARG encoding genes of the plants or plants cells, as described e.g. in WO 2004/090140.

3) a transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP 04077624.7, WO 2006/133827, PCT/EP07/002433, EP 1999263, or WO 2007/107326.

Enzymes involved in carbohydrate biosynthesis include those described in e.g. EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, EP 06090134.5, EP 06090228.5, EP 06090227.7, EP 07090007.1, EP 07090009.7, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. No. 5,824,790, U.S. Pat. No. 6,013,861, WO 94/04693, WO 94/09144, WO 94/11520, WO 95/35026 or WO 97/20936 or enzymes involved in the production of polyfructose, especially of the inulin and levan-type, as disclosed in EP 0663956, WO 96/01904, WO 96/21023, WO 98/39460, and WO 99/24593, the production of alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, the production of alpha-1,6 branched alpha-1,4-glucans, as disclosed in WO 00/73422, the production of alternan, as disclosed in e.g. WO 00/47727, WO 00/73422, EP 06077301.7, U.S. Pat. No. 5,908,975 and EP 0728213, the production of hyaluronan, as for example disclosed in WO 2006/032538, WO 2007/039314, WO 2007/039315, WO 2007/039316, JP 2006304779, and WO 2005/012529.

The nucleic acid molecule of interest may also comprise a selectable or screenable marker gene, which may or may not be removed after insertion, e.g as described in WO 06/105946, WO08/037436 or WO08/148559, to facilitate the identification of potentially correctly targeted events. Likewise, also the nucleic acid molecule encoding the DSBI enzyme may comprise a selectable or screenable marker gene, which preferably is different from the marker gene in the DNA of interest.

“Selectable or screenable markers” as used herein have their usual meaning in the art and include, but are not limited to plant expressible phosphinotricin acetyltransferase, neomycine phosphotransferase, glyphosate oxidase, glyphosate tolerant EPSP enzyme, nitrilase gene, mutant acetolactate synthase or acetohydroxyacid synthase gene, β-glucoronidase (GUS), R-locus genes, green fluorescent protein and the likes.

In one embodiment, the preselected site and/or cleavage site are located in the vicinity of an elite event, for example in one of the flanking region of the elite event, so that the modification that is introduced co-segregates with the elite locus, i.e. the modification and the elite event inherit as a single genetic unit, as e.g. described in WO2013026740. For this the preselected site preferably is located within 1 cM from the elite event locus, such as within 0.5 cM, within 0.1 cM, within 0.05 cM, within 0.01 cM, within 0.005 cM or within 0.001 cM from the elite event. Relating to base pairs, this can refer to within 5000 kb, within 1000 kb, within 500 kb, within 100 kb, within 50 kb, within 10 kb, within 5 kb, within 4 kb, within 3 kb, within 2 kb, within 1 kb, within 750 bp, within 500 bp, or within 250 bp from the existing elite event (depending on the species and location in the genome), e.g. between 0.5 kb and 10 kb or between 1 kb and 5 kb from the existing elite event. A list of elite events (including their flanking sequences) in the vicinity of which the genomic modification can be made according to the invention is given in table 1 of WO2013026740 on page 18-22, each of which is incorporated by reference herein).

The invention further provides the use of a DSBI enzyme (optionally in combination with a repair nucleic acid molecule as describe above) to modify the genome at a preselected site located at least at least 25 bp, at least 28 bp, at least 30 bp, at least 35 bp, at least 40 bp, at least 43 bp, at least 50 bp, at least 75 bp, at least 100 bp, at least 150 bp, at least 200 bp, at least 250 bp at least 300 bp, at least 400 bp, at least 500 bp, at least 750 bp, at least 1 kb, at least 1.5 kb, at least 2 kb, at least 3 kb, at least 4 kb, at least 5 kb, or at least 10 kb from the cleavage site of said DSBI enzyme. Said DSBI enzyme can be a DSBI enzyme that generates a 5′ overhang upon cleavage, or said DSBI enzyme can be a TALEN, particularly a TALEN generating a 5′ overhang, such as a TALEN with a FOKI nuclease domain.

In a further aspect, the invention provides a method for increasing the mutation frequency at a preselected site of the genome, preferably the nuclear genome, of a eukaryotic cell comprising the steps of:
a. Inducing a double stranded DNA break (DSB) in the genome of said cell at a cleavage site at or near a recognition site for a double stranded DNA beak inducing (DSBI) enzyme by expressing in said cell a DSBI enzyme inducing a DSB at said cleavage site;
b. Introducing into said cell a foreign nucleic acid molecule;
c. Selecting a cell wherein said DSB has been repaired resulting in a modification of said genome at said preselected site, wherein said modification is selected from;
   i. a replacement of at least one nucleotide;
   ii. a deletion of at least one nucleotide;
   iii. an insertion of at least one nucleotide; or
   iv. any combination of i.-iii.
characterised in that said foreign nucleic acid molecule also comprises a recognition site and cleavage site for said DSBI enzyme.

As used herein, a foreign nucleic acid molecule, can be a single stranded or double stranded DNA or RNA molecule, that also comprises a recognition site and cleavage site for the same DSBI enzyme that is used for inducing the genomic DSB, such that the repair nucleic acid molecule can also be cleaved by the DSBI enzyme inducing the genomic break. Again, it is believed that the cleavage of the foreign nucleic acid molecule enhances the recruitment of cellular enzymes involved in DNA repair and hence also enhances repair of the genomic DSB, thereby increasing the mutation frequency at the genomic cleavage site (i.e. the preselected site).

In one embodiment, the foreign nucleic acid molecule comprise a nucleotide sequence homologous to the genomic DNA region in the proximity of or comprising the recognition and/or cleavage site of the DSBI enzyme. The foreign nucleic acid molecule should preferably be at least 20 nt in length and have at least 80%, at least 90%, at least 95% or 100% sequence identity over at least 20 nt to the genomic DNA region in the proximity of or comprising the recognition and/or cleavage site. In the proximity of can be within about 10000 bp from the recognition and/or cleavage site, such as within about 5000 bp, about 2500 bp, about 1000 bp, about 500 bp, about 250 bp, about 100 bp, about 50 bp or about 25 bp from the recognition and/or cleavage site.

The DSBI enzyme according to this aspect can be any DSBI enzyme as described elsewhere in the application, including e.g. a TALEN, a ZFN, a Cas9 nuclease or a homing endonuclease (meganuclease), and can also be expressed in the cell as described elsewhere in the application. The foreign nucleic acid molecule can be introduced into the cell like any other nucleic acid molecule, also as described elsewhere in the application.

It will be appreciated that the methods of the invention can be applied to any eukaryotic organism, such as but not limited to plants, fungi, and animals, such as insects, nematodes, fish, and mammals. Accordingly, the eukaryotic cell can e.g. be plant cell, a fungal cell, or an animal cell, such as an insect cell, a nematode cell, a fish cell, and a mammalian cell.

The methods can be ex vivo or in vitro methods, especially when involving animals such as humans.

Plants (Angiospermae or Gymnospermae) include for example cotton, canola, oilseed rape, soybean, vegetables, potatoes, Lemna spp., Nicotiana spp., Arabidopsis, alfalfa, barley, bean, corn, cotton, flax, millet, pea, rape, rice, rye, safflower, sorghum, soybean, sunflower, tobacco, turfgrass, wheat, asparagus, beet and sugar beet, broccoli, cabbage, carrot, cauliflower, celery, cucumber, eggplant, lettuce, onion, oilseed rape, pepper, potato, pumpkin, radish, spinach, squash, sugar cane, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon.

It is also an object of the invention to provide eukaryotic cells that have a modification in the genome obtained by the methods of the invention, e.g. a plant cell, a fungal cell, or an animal cell, such as an insect cell, a nematode cell, a fish cell, mammalian cells and (non-human) stem cells.

In one embodiment, also provided are plant cells, plant parts and plants generated according to the methods of the invention, such as fruits, seeds, embryos, reproductive tissue, meristematic regions, callus tissue, leaves, roots, shoots, flowers, fibers, vascular tissue, gametophytes, sporophytes, pollen and microspores, which are characterised in that they comprise a specific modification in the genome (insertion, replacement and/or deletion). Gametes, seeds, embryos, either zygotic or somatic, progeny or hybrids of plants comprising the DNA modification events, which are produced by traditional breeding methods, are also included within the scope of the present invention. Such plants may contain a nucleic acid molecule of interest inserted at or instead of a target sequence or may have a specific DNA sequence deleted (even single nucleotides), and will only be different from their progenitor plants by the presence of this heterologous DNA or DNA sequence or the absence of the specifically deleted sequence (i.e. the intended modification) post exchange.

In particular embodiments the plant cell described herein is a non-propagating plant cell, or a plant cell that cannot be regenerated into a plant, or a plant cell that cannot maintain its life by synthesizing carbohydrate and protein from the inorganics, such as water, carbon dioxide, and inorganic salt, through photosynthesis.

The invention further provides a method for producing a plant comprising a modification at a predefined site of the genome, comprising the step of crossing a plant generated according to the above methods with another plant or with itself and optionally harvesting seeds.

The invention further provides a method for producing feed, food or fiber comprising the steps of providing a population of plants generated according to the above methods and harvesting seeds.

The plants and seeds according to the invention may be further treated with a chemical compound, e.g. if having tolerance to such a chemical.

Accordingly, the invention also provides a method of growing a plant generated according to the above methods, comprising the step of applying a chemical to said plant or substrate wherein said plant is grown.

Further provided is a process of growing a plant in the field comprising the step of applying a chemical compound on a plant generated according to the above methods.

Also provided is a process of producing treated seed comprising the step applying a chemical compound, such as the chemicals described above, on a seed of plant generated according to the above described methods.

The DSBI enzyme can be expressed in the cell by e.g. introducing the DSBI peptide directly into the cell. This can be done e.g. via mechanical injection, electroporation, the bacterial type III secretion system, or Agrobacterium mediated transfer (for the latter see e.g. Vergunst et al., 2000, Science 290: p 979-982). The DSBI enzyme can also be expressed in the cell by introducing into the cell a nucleic acid encoding the DSBI enzyme (e.g. a single stranded or double stranded RNA or DNA molecule), such as an mRNA which when translated results in the expression of the DSBI enzyme or a chimeric gene wherein a coding region for the DSBI enzyme is operably linked to a promoter driving expression in the host cell and optionally a 3′ end region involved in transcription termination and polyadenylation.

Nucleic acid molecules used to practice the invention, including the repair and foreign nucleic acid molecule as well as nucleic acid molecules encoding the DSBI enzyme, may be introduced (either transiently or stably) into the cell by any means suitable for the intended host cell, e.g. viral delivery, bacterial delivery (e.g. Agrobacterium), polyethylene glycol (PEG) mediated transformation, electroporation, vacuum infiltration, lipofection, microinjection, biolistics, virosomes, liposomes, immunoliposomes, polycation or lipid:nucleic acid conjugates, naked DNA, artificial virions, and calcium-mediated delivery.

Transformation of a plant means introducing a nucleic acid molecule into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation.

Transformed plant cells can be regenerated into whole plants. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

A nucleic acid molecule can also be introduced into a plant by means of introgression. Introgression means the integration of a nucleic acid in a plant's genome by natural means, i.e. by crossing a plant comprising the chimeric gene described herein with a plant not comprising said chimeric gene. The offspring can be selected for those comprising the chimeric gene.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

A chimeric gene, as used herein, refers to a gene that is made up of heterologous elements that are operably linked to enable expression of the gene, whereby that combination is not normally found in nature. As such, the term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism).

The expression “operably linked” means that said elements of the chimeric gene are linked to one another in such a way that their function is coordinated and allows expression of the coding sequence, i.e. they are functionally linked. By way of example, a promoter is functionally linked to another nucleotide sequence when it is capable of ensuring transcription and ultimately expression of said other nucleotide sequence. Two proteins encoding nucleotide sequences, e.g. a transit peptide encoding nucleic acid sequence and a nucleic acid sequence encoding a second protein, are functionally or operably linked to each other if they are connected in such a way that a fusion protein of first and second protein or polypeptide can be formed.

A gene, e.g. a chimeric gene, is said to be expressed when it leads to the formation of an expression product. An expression product denotes an intermediate or end product arising from the transcription and optionally translation of the nucleic acid, DNA or RNA, coding for such product, e.g. the second nucleic acid described herein. During the transcription process, a DNA sequence under control of regulatory regions, particularly the promoter, is transcribed into an RNA molecule. An RNA molecule may either itself form an expression product or be an intermediate product when it is capable of being translated into a peptide or protein. A gene is said to encode an RNA molecule as expression product when the RNA as the end product of the expression of the gene is, e.g., capable of interacting with another nucleic acid or protein. Examples of RNA expression products include inhibitory RNA such as e.g. sense RNA (co-suppression), antisense RNA, ribozymes, miRNA or siRNA, mRNA, rRNA and tRNA. A gene is said to encode a protein as expression product when the end product of the expression of the gene is a protein or peptide.

A nucleic acid or nucleotide, as used herein, refers to both DNA and RNA. DNA also includes cDNA and genomic DNA. A nucleic acid molecules can be single- or double-stranded, and can be synthesized chemically or produced by biological expression in vitro or even in vivo.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

The following non-limiting Examples describe the use of repair molecules for introducing targeted genomic modifications away from the cleavage site of TALENs.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

All patents, patent applications, and publications or public disclosures (including publications on internet) referred to or cited herein are incorporated by reference in their entirety.

The sequence listing contained in the file named “BCS13-2005-WO_ST25”, which is 95 kilobytes (size as measured in Microsoft Windows®), contains 13 sequences SEQ ID NO: 1 through SEQ ID NO: 13, is filed herewith by electronic submission and is incorporated by reference herein.

The invention will be further described with reference to the examples described herein; however, it is to be understood that the invention is not limited to such examples.

### SEQUENCE LISTING

Throughout the description and Examples, reference is made to the following sequences:

SEQ ID NO. 1: Nucleotide sequence of vector pT1B235

SEQ ID NO. 2: Nucleotide sequence of vector pTCV224

SEQ ID NO. 3: Nucleotide sequence of vector pTCV225

SEQ ID NO. 4: Nucleotide sequence of vector pTJR21

SEQ ID NO. 5: Nucleotide sequence of vector pTJR23

SEQ ID NO. 6: Nucleotide sequence of vector pTJR25

SEQ ID NO. 7: Nucleotide sequence of the bar gene (355-bar-3′nos)

SEQ ID NO. 8: Repair DNA vector pJR19

SEQ ID NO. 9: Primer IB448

SEQ ID NO. 10: Primer mdb548

SEQ ID NO. 11: Primer AR13

SEQ ID NO. 12: Primer AR32

SEQ ID NO. 13: Primer AR35

### EXAMPLES

### Example 1

### Vector construction

Using standard molecular biology techniques, the following vectors were created, containing the following operably linked elements:
Foreign/repair DNA vector pT1B235 (Seq ID No: 1):
   RB (nt 7946 to 7922): right border repeat from the T-DNA of Agrobacterium tumefaciens (Zambryski, 1988)
   Pcvmv (nt 8002 to 8441): sequence including the promoter region of the Cassava Vein Mosaic Virus (Verdaguer et al., 1996)
   5′cvmv (nt 8442 to 8514): 5′leader sequence from CsVMV gene
   Hyg-1 Pa (nt 8521 to 9546): hygromycin B phosphotransferase gene isolated from the E. coli plasmid pJR225 derived originally from Klebsiella. Gene provides resistance to aminoglycoside antibiotic hygromycin
   3′35S (nt 9558 to 9782): sequence including the 3′ untranslated region of the 35S transcript of the Cauliflower Mosaic Virus (Sanfaçon et al., 1991)
   LB (9885 to 9861): Left border repeat from the T-DNA of Agrobacterium tumefaciens (Zambryski, 1988)
Foreign/repair DNA vector pTCV224 (SEQ ID NO: 2):
   RB (nt 2 to 11322): right border repeat from the T-DNA of Agrobacterium tumefaciens (Zambryski, 1988)
   3′nos (nt 286 to 26): sequence including the 3′ untranslated region of the nopaline synthase gene from the T-DNA of pTiT37 (Depicker et al., 1982)
   bar(141-552) (nt 717 to 306): 5′ deletion coding sequence of bar-gene (coding sequence of the phosphinothricin acetyltransferase gene of Streptomyces hygroscopicus as described by Thompson et al. (1987)), deletion until base n° 140
   PCsVMV XYZ (747 to 1259): sequence including the promoter region of the Cassava Vein Mosaic Virus (Verdaguer et al., 1996)
   5′csvmv (nt 1187 to 1259): 5′leader sequence from CsVMV gene
   hyg-1 Pa (nt 1266 to 2291): hygromycin B phosphotransferase gene isolated from the E. coli plasmid pJR225 derived originally from Klebsiella. Gene provides resistance to aminoglycoside antibiotic hygromycin
   3′35S (nt 2303 to 2527): sequence including the 3′ untranslated region of the 35S transcript of the Cauliflower Mosaic Virus (Sanfaçon et al., 1991)
   bar(1-144) (nt 2672 to 2529): 3′ deletion coding sequence of bar-gene (coding sequence of the phosphinothricin acetyltransferase gene of Streptomyces hygroscopicus as described by Thompson et al. (1987)), deletion from base n° 145
   P35S3 (nt 3359 to 2673): sequence including the promoter region of the Cauliflower Mosaic Virus 35S transcript (Odell et al., 1985) (truncated as compared to target line, such that it cannot be recognized by primer IB448)
   LB (nt 3400 to 3376): left border repeat from the T-DNA of Agrobacterium tumefaciens (Zambryski, 1988)
Foreign/repair DNA vector pTCV225 (SEQ ID NO: 3):
   RB (nt 33 to 9): Right border repeat from the T-DNA of Agrobacterium tumefaciens (Zambryski, 1988)
   3′nos (nt 317 to 57): A fragment of the 3′ untranslated end of the nopaline synthase gene from the T-DNA of pTiT37 and containing plant polyadenylation signals (Depicker et al., 1982)
   bar(476-552) (nt 413 to 337): 5′ deletion coding sequence of bar-gene (coding sequence of the phosphinothricin acetyltransferase gene of Streptomyces hygroscopicus as described by Thompson et al. (1987)), deletion till base n° 476
   Pcsvmv XYZ (nt 443 to 882): Promoter of the cassava vein mosaic virus (Verdaguer et al., 1996)
   5′csvmv (nt 883 to 955): 5′leader sequence from CsVMV gene
   Hyg-1 Pa (nt 962 to 1987): hygromycin B phosphotransferase gene isolated from the E. coli plasmid pJR225 derived originally from Klebsiella. Gene provides resistance to aminoglycoside antibiotic hygromycin
   3′35S (nt 1999 to 2223): A fragment of the 3′ untranslated region of the 35S gene from the Cauliflower Mosaic Virus
   bar(1-479) (nt 2702 to 2224): 3′ deletion coding sequence of bar-gene (coding sequence of the phosphinothricin acetyltransferase gene of Streptomyces hygroscopicus as described by Thompson et al. (1987)), deletion from base n° 479
   P35S3 (nt 3389 to 2703): Fragment of the promoter region from the Cauliflower Mosaic Virus 35S transcript (Odell et al., 1985) (truncated as compared to target line, such that it cannot be recognized by primer IB448)
   LB (nt 3430 to 3406): left border repeat from the T-DNA of Agrobacterium tumefaciens (Zambryski, 1988)
Repair DNA vector pTJR21 (SEQ ID NO: 4):
   RB (nt 1 to 25): right border repeat from the T-DNA of Agrobacterium tumefaciens (Zambryski, 1988)
   3′nos (nt 309 to 49): sequence including the 3′ untranslated region of the nopaline synthase gene from the T-DNA of pTiT37 (Depicker et al., 1982)
   bind site (nt 540 to 522): bind site for TALE nuclease
   ½ spacer (nt 546 to 541): ½ spacer for TALE nuclease
   bar(335-552 bp) (nt 546 to 329): 5′ deletion coding sequence of bar-gene (coding sequence of the phosphinothricin acetyltransferase gene of Streptomyces hygroscopicus as described by Thompson et al. (1987)), deletion till base n° 334
   Pcsvmv XYZ (nt 576 to 1087): sequence including the promoter region of the Cassava Vein Mosaic Virus (Verdaguer et al., 1996)
   5′csvmv (nt 1016 to 1088): 5′leader sequence from CsVMV gene hyg-1 Pa (nt 1095 to 2120): hygromycin B phosphotransferase gene isolated from the E. coli plasmid pJR225 derived originally from Klebsiella. Gene provides resistance to aminoglycoside antibiotic hygromycin
   3′35S (nt 2132 to 2356): sequence including the 3′ untranslated region of the 35S transcript of the Cauliflower Mosaic Virus (Sanfaçon et al., 1991)
   ½ spacer (nt 2363 to 2358): ½ spacer for TALE nuclease
   bind site (nt 2382 to 2364): bind site for TALE nuclease
   bar(1-334 bp) (nt 2691 to 2358): 3′ deletion coding sequence of bar-gene (coding sequence of the phosphinothricin acetyltransferase gene of Streptomyces hygroscopicus as described by Thompson et al. (1987)), deletion from base n° 335
   P35S3 (nt 3378 to 2692): sequence including the promoter region of the Cauliflower Mosaic Virus 35S transcript (Odell et al., 1985) (truncated as compared to target line, such that it cannot be recognized by primer IB448)
   LB (nt 3395 to 3419): left border repeat from the T-DNA of Agrobacterium tumefaciens (Zambryski, 1988)
Repair DNA vector pTJR23 (SEQ ID NO: 5):
   RB (nt 1 to 25): right border repeat from the T-DNA of Agrobacterium tumefaciens (Zambryski, 1988)
   3′nos (nt 309 to 49): sequence including the 3′ untranslated region of the nopaline synthase gene from the T-DNA of pTiT37 (Depicker et al., 1982)
   bar(341-552 bp) (nt 540 to 329): 5′ deletion coding sequence of bar-gene (coding sequence of the phosphinothricin acetyltransferase gene of Streptomyces hygroscopicus as described by Thompson et al. (1987)), deletion till base n° 340
   bind site (nt 540 to 522): bind site for TALE nuclease
   Pcsvmv XYZ (nt 570 to 1081): sequence including the promoter region of the Cassava Vein Mosaic Virus (Verdaguer et al., 1996)
   5′csvmv (nt 1010 to 1082): 5′leader sequence from CsVMV gene
   hyg-1 Pa (nt 1089 to 2114): hygromycin B phosphotransferase gene isolated from the E. coli plasmid pJR225 derived originally from Klebsiella. Gene provides resistance to aminoglycoside antibiotic hygromycin
   3′35S (nt 2126 to 2350): sequence including the 3′ untranslated region of the 35S transcript of the Cauliflower Mosaic Virus (Sanfaçon et al., 1991)
   bind site (nt 2370 to 2352): bind site for TALE nuclease bar(1-328) (nt 2679 to 2352): 3′ deletion coding sequence of bar-gene (coding sequence of the phosphinothricin acetyltransferase gene of Streptomyces hygroscopicus as described by Thompson et al. (1987)), deletion from base n° 329
   P35S3 (nt 3366 to 2680): sequence including the promoter region of the Cauliflower Mosaic Virus 35S transcript (Odell et al., 1985)
   LB (nt 3383 to 3407): left border repeat from the T-DNA of Agrobacterium tumefaciens (Zambryski, 1988)
Repair DNA vector pTJR25 (SEQ ID NO: 6):
   RB (nt 1 to 25): Right border repeat from the T-DNA of Agrobacterium tumefaciens (Zambryski, 1988)
   3′nos (nt 309 to 49): sequence including the 3′ untranslated region of the nopaline synthase gene from the T-DNA of pTiT37 (Depicker et al., 1982)
   bar(360-552 bp) (nt 521 to 329): 5′ deletion coding sequence of bar-gene (coding sequence of the phosphinothricin acetyltransferase gene of Streptomyces hygroscopicus as described by Thompson et al. (1987)), deletion till base n° 359
   Pcsvmv XYZ (nt 551 to 1062): sequence including the promoter region of the Cassava Vein Mosaic Virus (Verdaguer et al., 1996)
   5′csvmv (nt 991 to 1062): 5′leader sequence from CsVMV gene
   hyg-1 Pa (nt 1070 to 2095): coding sequence of the hygromycin B phosphotransferase gene isolated from Klebsiella. Gene provides resistance to aminoglycoside antibiotic hygromycin
   3′35S (nt 2107 to 2331): sequence including the 3′ untranslated region of the 35S transcript of the Cauliflower Mosaic Virus (Sanfaçon et al., 1991)
   bar(1-309) (nt2641 to 2333): 3′ deletion coding sequence of bar-gene (coding sequence of the phosphinothricin acetyltransferase gene of Streptomyces hygroscopicus as described by Thompson et al. (1987)), deletion from base n° 310
   P35S3 (nt 3328 to 2642): sequence including the promoter region of the Cauliflower Mosaic Virus 35S transcript (Odell et al., 1985)
   LB (nt 3345 to 3369): Left border repeat from the T-DNA of Agrobacterium tumefaciens (Zambryski, 1988)
TALEN expression vector pTALENbar86 was developed comprising two chimeric genes, each of which encodes a TALEN monomer, operably linked to a constitutive promoter and universal terminator:
   Monomer 1: N-terminally and C-terminally truncated (Mussulino et al, 2011, Nucl Acids Res 9: p 9283-9293) artificial TAL effector with specific binding domain for sequence CTGCACCATCGTCAACCA (i.e. nt 903-920 of SEQ ID NO: 7) fused to the FOKI endonuclease cleavage domain
   Monomer 2: N-terminally and C-terminally truncated (Mussulino et al, 2011, supra) artificial TAL effector with specific binding domain for sequence ACGGAAGTTGACCGTGCT (i.e. nt 949-903 of SEQ ID NO: 7) fused to the FOKI endonuclease cleavage domain
Together TALENbar86 thus recognizes the nucleotide sequence 5′-CTGCACCATCGTCAACCA(N)₁₃AGCACGGTCAACTTCCCT-3′ (corresponding to nt 903-949 of seq ID NO: 7).
TALEN expression vector pTALENbar334 was developed comprising two chimeric genes, each of which encodes a TALEN monomer, operably linked to a constitutive promoter and universal terminator:
   Monomer 1: N-terminally and C-terminally truncated (Mussulino et al, 2011, supra) artificial TAL effector with specific binding domain for sequence CCACGCTCTACACCCACC (i.e. nt 1151-1168 of SEQ ID NO: 7) fused to the FOKI endonuclease cleavage domain
   Monomer 2: N-terminally and C-terminally truncated (Mussulino et al, 2011, supra) artificial TAL effector with specific binding domain for sequence TGAAGCCCTGTGCCTCCA (i.e. nt 1198-1181 of SEQ ID NO: 7) fused to the FOKI endonuclease cleavage domain
Together TALENbar334 thus recognizes the nucleotide sequence CCACGCTCTACACCCACC(N)₁₂TGGAGGCACAGGGCTTCA (corresponding to nt 1151-1198 of seq ID NO: 7).

### Example 2

### Plant Transformation

A PPT-resistant Tobacco target line was generated comprising a single copy of the bar gene operably linked to a 35S promoter and a nos terminator (SEQ ID NO: 7, p 35S: nt 1-840, bar coding region: nt 841-1392, 3′nos: nt 1411-1671).

Hemizygous protoplasts of the target line were transformed with the TALEN vectors and foreign/repair DNA vectors of Example 1 via electroporation.

### Example 3

### Mutation Induction by Bar-TALENs

Two TALENs cleaving the bar gene at position 86 and 334 respectively were evaluated for their cleavage efficiency in vivo, by transforming PPT-resistant target plants comprising a single copy functional bar gene with a bar-TALEN encoding vector (pTALENbar86 orpTALENbar334) together with a separate vector comprising a chimeric gene conferring hygromycin-resistance gene to be able to select transformants. Thus obtained hygromycin-resistant transformants were screened for PPT-sensitivity, indicating TALEN-mediated cleavage of the target site resulting in inactivation of the bar gene.

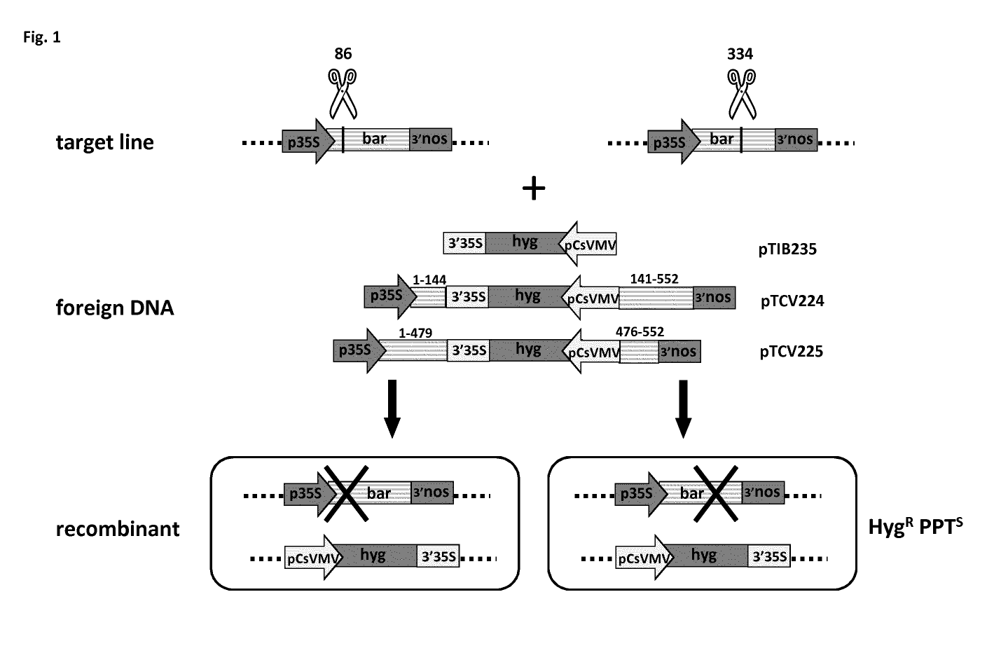

Three types of hygromycin cassettes were co-transformed with the TALEN vectors; pT1B235 not comprising flanking regions with homology to the DNA regions surrounding the target site, pTCV224 wherein the hyg-cassette is flanked with sequences homologous to the bar gene at nucleotide position 144, and pTCV225 wherein the hyg-cassette is flanked with sequences homologous to the bar gene at nucleotide position 479 (see FIG. 1 for a schematic representation). Table 1 depicts the % mutation induction that was observed for each of the combinations.

TABLE 1
mutation induction by bar-TALENS

| | Foreign | No. HygR | of which | % |
|---|---|---|---|---|
| TALEN | DNA | calli | pptS | mutation |
| pTALENbar86 | pTIB235 | 288 | 18 | 6.25 |
| | pTCV224 | 336 | 66 | 19.6 |
| | pTCV225 | 360 | 92 | 25.6 |
| pTALENbar334 | pTIB235 | 428 | 327 | 76 |
| | pTCV224 | 230 | 217 | 94.35 |
| | pTCV225 | 254 | 239 | 94.09 |

Surprisingly, in cases where the foreign DNA comprised the hyg cassette flanked with bar sequences which comprise the TALEN recognition sequence, the percentage of mutation induction was higher, up to a factor 3 to 4 for the lower performing TALENbar86 and up to nearly “saturation” for the higher performing TALENbar334, than in the absence of such flanking sequences. Presumably, this is due to the increased recruitment of DNA repair enzymes to the cleavage site in the foreign DNA, thereby also enhancing repair of the genomic DSB and increasing the mutation frequency at the genomic cleavage site.

### Example 4

### Targeted Insertion Using Bar-TALENs

Homology-mediated insertion at the TALEN target site

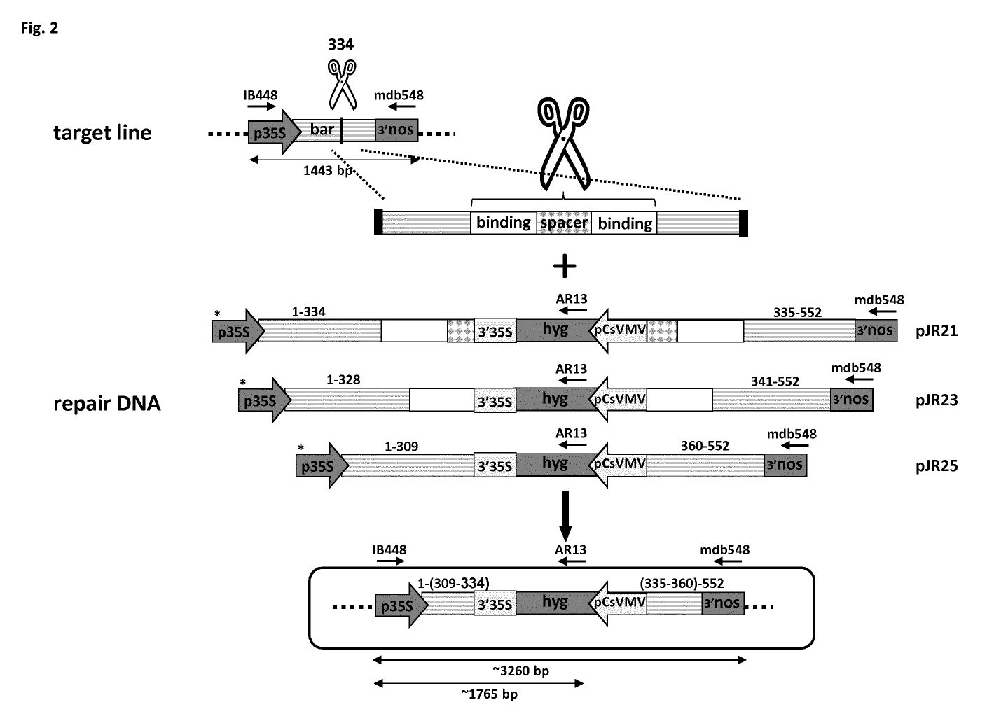

First, TALEN-driven targeted insertion at the target site was evaluated by co-transformation of the target line with pTALENbar334 and a repair DNA comprising a hyg-cassette with flanking regions homologous to the DNA regions flanking the cleavage site. Different flanking regions were designed, as schematically depicted in FIG. 2. The flanking regions of repair DNA vector pJR21 comprised sequences corresponding to half of the spacer region of the TALEN recognition site, sequences corresponding to the TALEN binding site and sequences corresponding to the bar gene. Repair DNA vector pJR23 is similar, except that it does not contain sequences corresponding to the spacer region, while repair DNA vector pJR25 lacks both the spacer and binding site sequences but contains the bar gene sequences.

Insertion of the hyg cassete at the target site was confirmed by PCR analysis of Hyg-resistant and PPT-sensitive calli using primer pairs 18448×mdb548 and 18448×AR13 (see FIG. 2). Note that due to a shorter 35S promoter in the repair DNAs, primer IB448 is not able to recognize the 35S promoter in the repair DNA (as indicated by the asterisk in FIG. 2), thereby allowing specific recognition of only the genomic 35S promoter from the target line. A shift in the size of PCR product from 1443 bp to 3257 bp with primer combination IB448×mdb548 and a PCR product of −1765 bp with the primer combination IB448×AR13 is indicative for homologous recombination-mediated insertion of the hyg gene at the target site. The percentage of correct targeted sequence insertion (TSI) events based on PCR analysis is given in table 2.

TABLE 2
homology-mediated insertion at TALEN target site
of TALENbar334

| | No. HygR | No. TSI | % |
|---|---|---|---|
| Repair DNA | calli | (PCR) | TSI |
| pTJR21 | 430 | 6 | 1.4 |
| pTJR23 | 573 | 10 | 1.8 |
| pTJR25 | 287 | 8 | 2.8 |

Thus, it appears that the insertion frequency is increased when choosing the homology sequences to not immediately flank the break site/or not to include sequences from the recognition site and/or cleavage site.

Sequence analysis of the upstream and downstream junctions of individual TSI events revealed that the junction at the side of pCsVMV (i.e. downstream of the cleavage site, relative to the transcriptional direction of the bar gene, see FIG. 2) always contained no sequence alterations (precise homologous recombination up to the nucleotide), whereas this was only the case for some of the junctions at the side of 3′35S (i.e upstream of the cleavage site, relative to the transcriptional direction of the bar gene, see FIG. 2), where small deletions or insertions were sometimes observed (see Table 3). A similar asymmetry was observed for repair of a TALEN-induced break (Bedell et al, 2012, Nature 491, p 114-118) and repair of a ZNF-induced break (Qi et al., 2013, Genome Res ePub Jan. 2, 2013).

TABLE 3
Sequencing of upstream and downstream junctions
of TSI events at TALEN cleavage site

| Repair DNA | 3′35S junction | | pCsVMV junction |
|---|---|---|---|
| pTJR21 | del 12 | b | OK |
| | OK | | OK |
| | OK | | OK |
| | del 114 | bp | OK |
| | del 41 | bp | OK |
| pTJR23 | del 97 | bp | OK |
| | OK | | OK |
| | del 340 | bp | OK |
| | ins 80 | bp | OK |
| | OK | | OK |
| | OK | | OK |
| | ins 101 | bp | OK |
| | del 187 | bp | OK |
| pTJR25 | OK | | nd |
| | OK | | nd |
| | ins 274 | bp | nd |

| | |
|---|---|
| nd | OK |

Homology-Mediated Insertion Upstream or Downstream of the TALEN Recognition Site

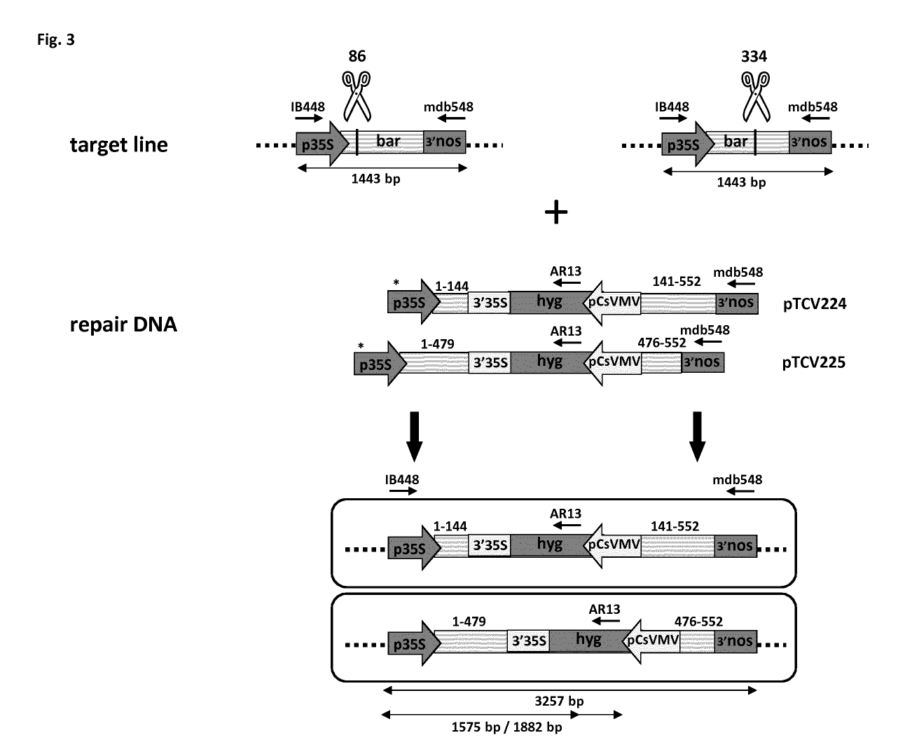

Next, TALEN-induced targeted insertion further away from the site of double stranded DNA break induction was evaluated by co-transformation with repair DNA vectors with flanking regions for targeted insertion either upstream or downstream of the break site, as is schematically depicted in FIG. 3. Repair DNA vector pTCV224 contained flanking sequences for insertion at nucleotide position 144 of the bar coding sequence, while repair DNA vector pTCV225 contained flanking sequences for insertion at position 479.

Insertion of the hyg cassete at the target site was again determined by PCR analysis of Hyg-resistant and PPT-sensitive calli using primer pairs 18448×mdb548 and 18448×AR13 (see FIG. 3). The percentage of candidate correct targeted sequence insertion (TSI) events based on PCR analysis is given in table 4.

TABLE 4
homology-mediated insertion away from TALEN cleavage
and recognition site

| | | | No. | No. | |
|---|---|---|---|---|---|
| | | | HygR | TSI | % |
| TALEN | repair DNA | Distance | calli | (PCR) | TSI |
| pTALENbar86 | pTCV224 (144) | +58 bp | 65 | 3 | 4.6 |
| | pTCV225 (479) | +393 bp | 92 | 4 | 4.3 |
| pTALENbar334 | pTCV224 (144) | −190 bp | 152 | 1 | 0.7 |
| | pTCV225 (479) | +145 bp | 217 | 15 | 6.9 |

It was surprisingly found that with values ranging from 4.3 to 6.9%, the frequency of homology-mediated TSI downstream (relative to the transcriptional direction of the bar gene) of the TALEN recognition site was about 2-4× as efficient as insertion at the recognition site (1.4-2.8%), whereas TSI upstream of the recognition site was decreased and up to 10× less efficient as downstream of the recognition site (0.7%). This difference in TSI frequency at one side of the break compared to at the other side might be related to differences in DNA binding affinity of the two TALEN monomers making up a functional TALEN dimer and might be reversed for other enzymes.

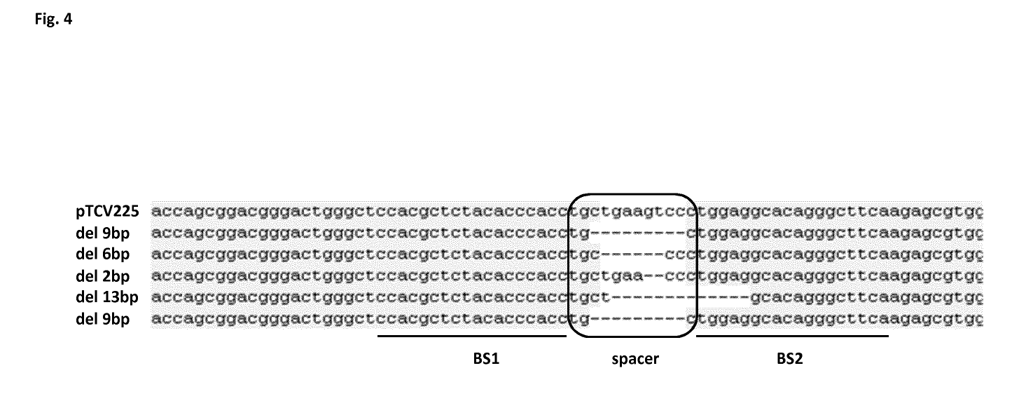

Sequence analysis of individual recombinant events with TALENbar334 and ptCV225 revealed perfect HR-mediated insertion of the hyg cassette at position 479 in the bar gene, but small deletions (from 2 to 13 bp) at the TALEN cleavage site, indicating repair by HR at one side of the DSB and repair by NHR at the other side of the DSB. (see Table 5). An alignment of the deletions observed at the TALENbar334 cleavage site after insertion of repair DNA pTCV225 is depicted in FIG. 4. These small deletions at the cleavage site are often unique for each event, and can thus be used as a footprint allowing discrimination and tracing of specific events.

TABLE 5
Sequencing of the cleavage site of TSI events
outside the TALEN cleavage site

| | | TALEN | |
|---|---|---|---|
| | | cleavage | |
| TALEN | Repair DNA | site | |
| pTALENbar86 | pTCV224 | OK | |
| | | del 5 | bp |
| | | del 5 | bp |
| | pTCV225 | ins 96 | bp |
| | | nd | |
| | | OK | |
| | | del 2 | bp |
| pTALENbar334 | pTCV224 | OK | |
| | pTCV225 | del 9 | bp |
| | | del 6 | bp |
| | | del 2 | bp |
| | | del 13 | bp |
| | | del 9 | bp |

For comparison, the target line was cotransformed with a vector encoding a bar meganuclease designed for cleavage at position 479 of the bar coding sequence (recognizing the target site GGGAACTGGCATGACGTGGGTTTC, i.e. nt 1306-1329 of SEQ ID NO. 7) together with repair DNA pTCV225 (for insertion at the cleavage site), resulting in a frequency of TSI events of 1.8% ( 3/164 hyg-resistant calli). Sequence analysis showed no sequence alterations at either the upstream or downstream junction, indicating perfect homology-mediated insertion at both sides.

### Example 5

### Allele Surgery Using Bar-TALENs

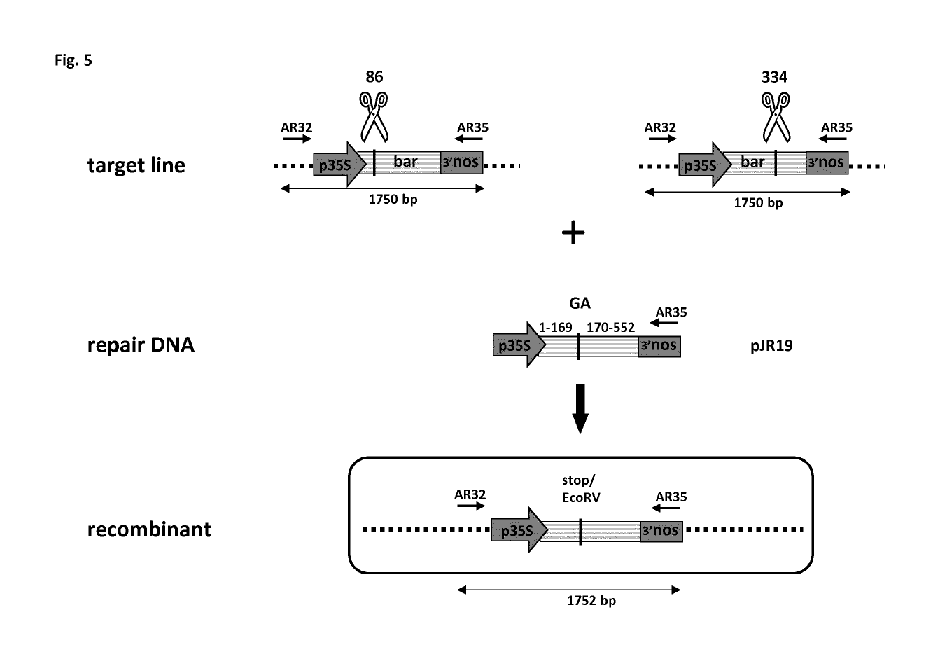

To test whether TALENs could also be used to make small targeted mutations of only one or several nucleotides away from the cleavage site, repair DNA vector pJR19 was designed to introduce a 2 bp insertion at position 169 of the bar gene, thereby creating a premature stop codon in the bar coding sequence and introducing an EcoRV site (FIG. 5).
Repair DNA vector pJR19 (SEQ ID NO: 8):
   P35S3 (nt 691 to 1543): sequence including the promoter region of the Cauliflower Mosaic Virus 35S transcript (Odell et al., 1985)
   bar-mut1 (nt 1544 to 2097): mutated coding sequence of bar gene (phosphinothricin acetyltransferase gene of Streptomyces hygroscopicus (Thompson et al. (1987)),mutation by insertion of GA at position n° 169-170 resulting in the creation of a pre-mature stop codon
   3′nos (nt 2117 to 2377): sequence including the 3′ untranslated region of the nopaline synthase gene from the T-DNA of pTiT37 (Depicker et al., 1982)

The target line was again co-transformed with either pTALENbar86 or pTALENbar334 together with repair DNA pJR19. PPT sensitive events (indicative for a mutation in the bar gene) were subjected to PCR analysis with primers AR32×A35 (see FIG. 5) and obtained PCR products were digested with EcoRV to identify perfect genome editing events. Again, modification downstream of the cleavage site was far more efficient than upstream. Out of the 150 PPT sensitive calli obtained when targeting downstream from the cleavage site, 6 events were found to contain the intended GA insertion as determined by EcoRV cleavage. When targeting upstream of the cleavage site, none of the 258 PPT sensitive calli contained the GA insertion (table 6).

TABLE 6
Homology-mediated allele surgery away from the TALEN
cleavage and recognition site

| | | | No. | | |
|---|---|---|---|---|---|
| | | | PPT^{S} | | % |
| TALEN | repair DNA | Distance | calli | PCR + EcoRV | TSI |
| pTALENbar86 | pJR19 (169) | +83 bp | 150 | 6 | 4.0 |
| pTALENbar334 | pJR19 (169) | −165 bp | 258 | 0 | 0.0 |

Of these 6 events, 5 were cloned and sequenced, and all 5 could be confirmed to contain the intended GA insertion. Of these, 4 events showed again small deletions (3-9 bp) but 1 event did not contain any mutations at the TALEN cleavage site. When for example editing in coding regions, such scars at the cleavage site could be prevented by introducing silent mutations in the recognition site for the DSBI enzyme in the repair molecule.

Taken together, TALENs appear a very efficient tool for making targeted mutations, especially when co-introducing a foreign nucleic acid molecule that can also be cleaved by the enzyme. TALENs are also very efficient for making targeted sequences insertions, including modification of only one or a few nucleotides (allele surgery), especially when designing the repair molecule for insertion/replacement further away from the cleavage site, i.e. outside of the cleavage and recognition site. This thus reduces the need to develop a particular enzyme—repair molecule combination for every intended genomic modification, thereby on the one hand thus allowing the use of one repair molecule with various enzymes to be evaluated for cleavage at a particular locus, while on the other hand allowing to make multiple targeted genomic modifications at a certain locus using only one enzyme in combination with various repair molecules.

1. A method for modifying the genome of a eukaryotic cell at a preselected site comprising the steps of:
a. Inducing a double stranded DNA break (DSB) in the genome of said cell at a cleavage site at or near a recognition site for a double stranded DNA break inducing (DSBI) enzyme by expressing in said cell a DSBI enzyme recognizing said recognition site and inducing a DSB at said cleavage site;
b. Introducing into said cell a repair nucleic acid molecule comprising an upstream flanking region having homology to the region upstream of said preselected site and/or a downstream flanking region having homology to the DNA region downstream of said preselected site for allowing homologous recombination between said flanking region or regions and said DNA region or regions flanking said preselected site;
c. Selecting a cell having a modification of said genome at said preselected site selected from
i. a replacement of at least one nucleotide;
ii. a deletion of at least one nucleotide;
iii. an insertion of at least one nucleotide; or
iv. any combination of i.-iii.
characterised in that said preselected is located outside said cleavage and/or recognition site.

2. The method of claim 1, wherein said preselected site is located at least 28 bp from said cleavage site.

3. The method of claim 1 or 2, wherein said preselected site is located at least 43 bp from said cleavage site

4. The method of any one of claims 1-3, wherein said repair molecule also comprises a recognition and cleavage site for said DSBI enzyme, preferably in one of said flanking regions.

5. The method of any one of claims 1-4, wherein said DSBI enzyme upon inducing said DSB creates a 5 overhang.

6. The method of any one of claims 1-5, wherein said DSBI enzyme is a TALEN.

7. The method of any one of claims 1-6, wherein said preselected site is located downstream of said recognition site.

8. The method of any one of claims 1-7, wherein said repair molecule is a double-stranded DNA molecule.

9. The method of any one of claims 1-8, wherein said repair molecule comprises a nucleic acid molecule of interest, said molecule of interest being inserted at said preselected through homologous recombination between said flanking DNA region or regions and said DNA region or regions flanking said preselected site.

10. The method of any one of claims 1-9, wherein said modification is a replacement or insertion of at least 43 nucleotides.

11. The method of any one of claims 1-10, wherein said DSBI enzyme is expressed in said cell by introducing into said cell a nucleic acid molecule encoding said DSBI enzyme.

12. The method of any one of claims 1-11, wherein said eukaryotic cell is a plant cell.

13. The method of any one of claims 1-12, wherein said nucleic acid molecule of interest comprises one or more expressible gene(s) of interest, said expressible gene of interest optionally being selected from the group of a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites.

14. The method of any one of claims 9-13, wherein said nucleic acid molecule of interest comprises a selectable or screenable marker gene.

15. The method of any one of claims 12-14, wherein said preselected site is located in the flanking region of an elite event.

16. The method of any one of claims 1-15, comprising the further step of growing said selected eukaryotic cell into a eukaryotic organism.

17. Use of a DSBI enzyme to modify the genome at a preselected site located outside the cleavage site and/or recognition site of said DSBI enzyme.

18. Use of claim 17, wherein said DSBI enzyme is a DSBI enzyme generating a 5 overhang upon cleavage, or wherein said DSBI enzyme is a TALEN or a ZFN.

19. A method for increasing the mutation frequency at a preselected site of the genome of a eukaryotic cell comprising the steps of:
a. Inducing a double stranded DNA break (DSB) in the genome of said cell at a cleavage site at or near a recognition site for a double stranded DNA break inducing (DSBI) enzyme by expressing in said cell a DSBI enzyme recognizing said recognition site and inducing a DSB at said cleavage site;
b. Introducing into said cell a foreign nucleic acid molecule;
c. Selecting a cell wherein said DSB has been repaired,
said repair of said double stranded DNA break resulting in a modification of said genome at said preselected site, wherein said modification is selected from;
i. a replacement of at least one nucleotide;
ii. a deletion of at least one nucleotide;
iii. an insertion of at least one nucleotide; or
iv. any combination of i.-iii.
characterised in that said foreign nucleic acid molecule also comprises a recognition site and cleavage site for said DSBI enzyme.

20. The method according to claim 19, wherein said foreign nucleic acid molecule comprises a nucleotide sequence of at least 20 nt in length having at least 80% sequence identity to a genomic DNA region within 5000 bp of said recognition and cleavage site.

21. A eukaryotic cell or eukaryotic organism, comprising a modification at a predefined site of the genome, obtained by the method of any one of claims 1-20.

22. A plant cell or plant comprising a modification at a predefined site of the genome, obtained by the method of any one of claims 1-20.
